(19) United States
(12) Patent Application Publication
(10) Pub. No.: US 2016/0244716 A1
(43) Pub. Date: Aug. 25, 2016

(54) PROTOCOL AND MEDIA FOR STORAGE AND TRANSPORT OF NK-92 CELL LINE

(71) Applicants: Hans G. KLINGEMANN, Culver City, CA (US); Barry J. SIMON, Culver City, CA (US); NANTKWEST, INC., Culver City, CA (US)

(72) Inventors: Hans G. Klingemann, Culver City, CA (US); Barry J. Simon, Culver City, CA (US)

(21) Appl. No.: 15/028,280

(22) PCT Filed: Oct. 7, 2014
(86) PCT No.: PCT/US2014/059551

§ 371 Date: Apr. 8, 2016

Related U.S. Application Data
(60) Provisional application No. 61/888,476, filed on Oct. 8, 2013.
(60) Provisional application No. 61/888,478, filed on Oct. 8, 2013.
(60) Provisional application No. 61/899,068, filed on Nov. 1, 2013.

(51) Int. Cl. C12N 5/00; C12N 5/0783

(52) U.S. Cl. CPC C12N 5/0018; C12N 5/0646; C12N 2501/2302; C12N 2523/00; C12N 2500/02

(57) ABSTRACT

Described herein is a storage medium for transport of NK-92 cells comprising human serum, about 200 IU/mL and a density of non-irradiated NK-92 cells sufficient to provide a therapeutic amount of NK-92 cells at the time of delivery to a treatment facility, wherein the temperature of the medium is maintained within +/−5° C. of a selected temperature between 20° C. and 40° C., such that the NK-92 cells remain viable for administration to a patient for up to a period of at least 24 hours after placement into the storage medium. Also described are methods of transporting NK-92 cells such that the NK-92 cells remain viable for administration to a patient for up to a period of at least 24 hours after placement into the storage medium.

### FIELD OF THE INVENTION

This invention relates to storage and transport of NK-92 cells such that the cells remain viable for administration to a patient, for example, for a period of at least 24 hours after placement into a storage medium.

### BACKGROUND OF THE INVENTION

Certain cells of the immune system have cytotoxic activity against particular target cells. Natural killer (NK) cells, generally representing about 10-15% of circulating lymphocytes, bind and kill target cells, including virus-infected cells and many malignant cells, nonspecifically with regard to antigen and without prior immune sensitization. Herberman et al., Science 214:24 (1981). Killing of target cells occurs by inducing cell lysis. NK cells have been shown to be effective in both ex vivo therapy and in vivo treatment in patients with advanced cancer. However, endogenous NK cells (i.e., those that are harvested from a donor or from the patient) remain difficult to work with and to apply in immunotherapy. It is difficult to expand NK cells ex vivo that maintain their tumor-targeting, tumoricidal, and viricidal capabilities in vivo, a major obstacle to their clinical use in adoptive cell immunotherapy. Melder, et al., Cancer Research 48:3461-3469 (1988); Stephen, et al., Leuk. Lymphoma 377-399 (1992); Rosenberg, et al., New Engl. J. Med. 316:889-897 (1987). Further, preparations of endogenous NK cells include T cells and/or other immune effector cells that must be removed if the NK cells are used to treat a patient unrelated to the donor.

Embodiments described herein generally relate to a storage medium for transport and/or delivery of NK-92 cells, as well as to methods of transporting and using the transported NK-92 cells.

### SUMMARY OF THE INVENTION

The NK-92 cell line is a unique cell line that was discovered to proliferate in the presence of interleukin 2 (IL-2). Gong et al., Leukemia 8:652-658 (1994). These cells have high cytolytic activity against a variety of cancers. The NK-92 cell line is a homogeneous NK cell population having broad anti-tumor cytotoxicity with predictable yield after expansion. Phase I clinical trials have confirmed its safety profile, and anti-tumor responses in certain patients with advanced cancer have been observed.

One limitation to treatment of patients with NK-92 cells is the ability to store and/or transport the cells over long periods of time. NK cells used in patient therapy must be maintained under current good manufacturing processes (cGMP), thereby limiting the number of facilities that can culture and prepare the NK-92 cell line. To date, use of NK-92 cells in patient therapy has been limited by the availability of culture and production facilities very near to the hospital where treatment is to occur; NK-92 cells are now prepared in a facility next to a hospital and then hand-carried to the medical team for use in treatment of a patient. Currently, frozen NK-92 cells exhibit dramatically reduced cytotoxicity after thawing. Even after a recovery period in culture of several days, NK-92 cell number and cytotoxicity remain suboptimal.

Heretofore, studies on endogenous NK cells have indicated that IL-2 (1000 IU/mL) is critical for NK cell activation during shipment, but that the cells need not be maintained at 37 ° C. and 5% carbon dioxide. Koepsell, et al., Transfusion 53:398-403 (2013). However, endogenous NK cells are significantly different from NK-92 cells, in large part because of their distinct origins: NK-92 is a cancer-derived cell line, whereas endogenous NK cells are harvested from a donor (or the patient) and processed for infusion into a patient. Endogenous NK cell preparations are heterogeneous cell populations, whereas NK-92 cells are a homogeneous, clonal cell line. NK-92 cells readily proliferate in culture while maintaining cytotoxicity, whereas endogenous NK cells do not. In addition, an endogenous heterogeneous population of NK cells does not aggregate at high density. Accordingly, maximization of the viability and cytotoxicity after storage and/or transportation of these two cell types requires very different considerations.

NK-92 cells present other problems. For example, NK-92 requires the presence of IL-2 to remain viable and to proliferate and will proliferate in as little as 1 IU/mL of IL-2. Gong, et al. supra. Moreover, the cellular morphology and/or phenotype changes with the amount of IL-2 used in the medium. Gong, et al. supra. For example, IL-2 receptor (CD-25) expression is inversely proportional to IL-2 concentration. Gong, et al. supra.

Still further, NK-92 cell aggregation at high concentrations negatively impacts their use in treating patients. This creates a conundrum in that in the absence of IL-2, the NK-92 cells lose viability, whereas, in the presence of IL-2, the cells proliferate. Indeed, under normal cell culture conditions, the number of NK-92 cells will double in about 26-32 hours. This conundrum is further complicated by the fact that the storage medium must be capable of permitting transport to hospitals for up to at least 24 hours from placement of the NK-92 cells into the storage medium. The rate of NK-92 cell proliferation at any given IL-2 concentration must be taken into account in order to prevent aggregation during transport.

A time frame of, for example, at least about 24 hours permits placement of the cells into the storage medium, transport to the hospital, and then implementation of proper procedures to inject the NK-92 cells into the patient. Once NK-92 cells arrive at the hospital (or other treatment facility), they are washed and transferred into a solution suitable for injection into a patient, e.g., phosphate buffered saline (PBS). NK-92 cells also are irradiated to prevent in vivo proliferation. Although the radiation dose is designed to maintain cytotoxicity, it adversely impacts viability of the cells. Accordingly, it is important that the storage medium used for transport maintain NK-92 cell viability to the greatest extent possible in order to minimize degradation of the cell population during conversion into a form useful for treatment.

There thus remains a need for a storage medium for transporting the NK-92 cell line such that the cells can be cultured and prepared at a central facility and distributed to a distant hospital or other treatment facility for treatment of a patient soon after receipt. This need encompasses a method that retains NK-92 cell viability and cytotoxicity during storage and/or transport while minimizing proliferation of the cells to the point where substantial aggregation occurs. This invention provides storage medium and methods of storing and/or transporting non-irradiated NK-92 cells that address these needs.

The present invention in some embodiments provides a storage medium and method for storing and/or transporting the NK-92 cell line in order to maintain viability and cytotoxicity of the cells for treatment of a patient at a facility distant from the facility of origin of the NK-92 cells. This invention is predicated on the surprising discovery that storing NK-92 cells in the presence of low levels of IL-2 and at a minimally variable temperature can limit proliferation yet prevent substantial aggregation due to increased cell density, while also maintaining cell viability and cytotoxicity. A central facility can prepare NK-92 cells for therapy, and then transport the cells under the conditions provided by the invention for therapeutic use in patients at remote treatment facilities, without a requirement for specialized cGMP compliant facilities at the treatment facility. The invention thus allows for a reproducible “off-the-shelf” NK-92 product with only minimal manipulation of the cells at the treatment facility. The invention further provides a method of delivering a homogeneous NK-92 cell product to a destination with consistent cell viability.

In one aspect, the invention described herein relates generally to a cell storage medium for transport and/or delivery of cells, for example, to a sterile, isotonic NK-92 cell storage medium for transport and delivery of NK-92 cells, the storage medium comprising: human serum or human serum albumin; an initial density of non-irradiated, substantially non-aggregated NK-92 cells sufficient to provide a therapeutic amount of NK-92 cells at the time of delivery to a treatment facility; and an IL-2 concentration of about 200 IU/mL wherein said medium is maintained within +/−5 ° C. of a temperature selected for transport, and further wherein said temperature selected for transport is between about 20° C. to about 40° C. in the presence of sufficient oxygen to maintain viability of the cells, such that the NK-92 cells remain viable for administration to a patient up to a period of at least 24 hours after placement into the storage medium. In a preferred embodiment, the therapeutic amount of NK-92 cells in the medium is no more than about 2.5×10 ̂6 cells/ml at the time of delivery to the treatment facility. In some embodiments, the NK-92 cell density is maintained between about 1×10 ̂4 cells/mL to about 1×10 ̂7 cells/mL during transport. In some embodiments, the NK-92 cell density is maintained between about 0.5×10 ̂6 cells/mL to about 8×10 ̂6 cells/mL during transport. In some embodiments, said medium is maintained within +/−2° C. of the temperature selected for transport. In some embodiments, the medium comprises about 1% to about 5% human serum or human serum albumin. In some embodiments, the temperature selected for transport is between about 25° C. to about 38° C. In some embodiments, the initial cell density is between about 1×10 ̂6 cells/ml and 6×10 ̂6 cells/ml.

In another aspect, the invention described herein relates to a method for transporting non-irradiated, substantially non-aggregated NK-92 cells to maintain viability of said NK-92 cells for administration to a patient for up to a period of at least 24 hours after placement into a storage medium, the method comprising:

(a) providing a sterile storage container;

(b) adding sterile storage medium to said container, wherein said medium comprises human serum or human serum albumin, and about 200 IU/mL of IL-2;

(c) adding to the container an initial density of non-irradiated, substantially non-aggregated NK-92 cells sufficient to provide a therapeutic amount of NK-92 cells at the time of delivery to a treatment facility;

(d) sealing the container;

(e) maintaining the medium within +/−5° C. of a temperature selected for transport, wherein said temperature selected for transport is between about 20° C. to about 40° C.; and

(f) transporting the NK-92 cells for up to a period of at least 24 hours after placement into the storage medium, in the presence of sufficient oxygen to maintain viability of the cells,

such that the NK-92 cells remain viable for administration to a patient for up to a period of at least 24 hours after placement into the storage medium.

In a preferred embodiment, the therapeutic amount of NK-92 cells in the medium is no more than about 2.5×10 ̂6 cells/ml at the time of delivery to the treatment facility. In some embodiments, the NK-92 cell density is maintained between about 1×10 ̂4 cells/mL to about 1×10 ̂7 cells/mL during transport. In some embodiments, the NK-92 cell density is maintained between about 0.5×10 ̂6 cells/mL to about 8×10 ̂6 cells/mL during transport. In some embodiments, said medium is maintained within +/−2° C. of the temperature selected for transport. In some embodiments, a temperature control device maintains the storage medium within +/−5° C. of the temperature selected for transport. In some embodiments, the storage container is labeled with the date and time the non-irradiated NK-92 cells were added to the medium. In some embodiments, the medium comprises about 1% to about 5% human serum or human serum albumin. In some embodiments, the temperature selected for transport is between about 25° C. to about 38° C. In some embodiments, the initial cell density is between about 1×10 ̂6 cells/ml and 6×10 ̂6 cells/ml.

### BRIEF DESCRIPTION OF THE DRAWINGS

FIG. 1 shows the doubling time of NK-92 cells in a growth culture.

FIG. 2A indicates the cytotoxicity of NK-92 cells shipped overnight at a density of 1×10 ̂6 cells/mL (red circles) or 6×10̂6 cells/mL (blue squares), or stored overnight in an incubator at a density of 4×10 ̂5 cells/mL (green triangles).

FIG. 2B indicates the cytotoxicity of the cells after dilution and resting.

FIG. 3 indicates the results of flow cytometry analysis of NK-92 cells shipped overnight at a density of 1×10 ̂6 cells/mL or 6×10 ̂6 cells/mL.

### DETAILED DESCRIPTION OF THE INVENTION

Before the present compositions and methods are disclosed and described, it is to be understood that the aspects described below are not limited to specific compositions, methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings^{.}

It must be noted that, as used in the specification and the appended claims, the singular forms “a,” “an” and “the” include plural referents unless the context clearly dictates otherwise.

As used herein, the term “about” means that a value may vary +/−15% and remain within the scope of the invention. For example, “an IL-2 concentration of about 200 IU/mL” encompasses an IL-2 concentration between 170 IU/mL and 230 IU/mL.

As used to describe the present invention, “natural killer (NK) cells” are cells of the immune system that kill target cells in the absence of a specific antigenic stimulus, and without restriction according to MHC class. Target cells may be tumor cells or cells harboring viruses. NK cells are characterized by the presence of CD56 and the absence of CD3 surface markers.

The term “endogenous NK cells” is used to refer to NK cells derived from a donor (or the patient), as distinguished from the NK-92 cell line. Endogenous NK cells are generally heterogeneous populations of cells within which NK cells have been enriched. Endogenous NK cells may be intended for autologous or allogeneic treatment of a patient.

The immortal NK cell line, NK-92, was originally obtained from a patient having non-Hodgkin's lymphoma. For purposes of this invention and unless indicated otherwise, the term “NK-92” is intended to refer to the original NK-92 cell lines as well as NK-92 cell lines that have been modified (e.g., by introduction of exogenous genes). NK-92 cells and exemplary and non-limiting modifications thereof are described in U.S. Pat. Nos. 7,618,817; 8,034,332; and 8,313,943, all of which are incorporated herein by reference in their entireties.

As used herein, “non-irradiated NK-92 cells” are NK-92 cells that have not been irradiated. Irradiation renders the cells incapable of growth and proliferation. It is envisioned that the NK-92 cells will be irradiated at the treatment facility or some other point prior to treatment of a patient, since the time between radiation and infusion should be no longer than four hours in order to preserve optimal activity. Alternatively, NK-92 cells may be inactivated by another mechanism.

As used to describe the present invention, “inactivation” of the NK-92 cells renders them incapable of growth and/or their normal function, in particular, their cytotoxic activity. Inactivation may also relate to the death of the NK-92 cells. It is envisioned that the NK-92 cells may be inactivated after they have effectively purged an ex vivo sample of cells related to a pathology in a therapeutic application, or after they have resided within the body of a mammal a sufficient period of time to effectively kill many or all target cells residing within the body. Inactivation may be induced, by way of nonlimiting example, by administering an inactivating agent to which the NK-92 cells are sensitive.

As used herein, the term “substantially non-aggregated” means that the NK-92 cell density is less than that which would have an adverse effect on aggregation. That is, the density of NK-92 cells is such that any aggregation does not materially alter their in vivo efficacy. Cellular density of about 1×10 ̂7 cells/mL is contemplated to be the upper limit of NK-92 cellular density while maintaining adequate viability and non-aggregation.

Materiality of the aggregation is measured by cell density of greater than about 1×10 ̂7 cells/mL. NK-92 cell densities during shipment which are less than about 1×10 ̂7 cell/mL will not materially alter their in vivo efficacy. As the NK-92 cells will proliferate in the storage medium, the amount of cells initially added to the storage medium must reflect the degree of growth and the length of time during which storage will be maintained. Such calculations are well within the skill of the art.

As used to describe the present invention, the terms “cytotoxic” and “cytolytic”, when used to describe the activity of effector cells such as NK cells, are intended to be synonymous. In general, cytotoxic activity relates to killing of target cells by any of a variety of biological, biochemical, or biophysical mechanisms. Cytolysis refers more specifically to activity in which the effector lyses the plasma membrane of the target cell, thereby destroying its physical integrity. This results in the killing of the target cell. Without wishing to be bound by theory, it is believed that the cytotoxic effect of NK cells is due to cytolysis.

As used to describe the present invention, “target cells” are the cells that are killed by the cytotoxic activity of the NK cells of the invention. These include in particular cells that are malignant or otherwise derived from a cancer, and cells that are infected by pathogenic viruses such as HIV, EBV, CMV, or herpes.

As used to describe the present invention, “purging” relates to killing of target cells by effector cells such as NK cells ex vivo. The target cells may be included in a biological sample obtained from a mammal believed to be suffering from a pathology related to the presence of the target cell in the sample. The pathology may be a cancer or malignancy due to tumor cells in the sample, and may be treated by purging the sample of the tumor cells and returning the sample to the body of the mammal.

As used to describe the present invention, “cancer”, “tumor”, and “malignancy” all relate equivalently to a hyperplasia of a tissue or organ. If the tissue is a part of the lymphatic or immune system, malignant cells may include non-solid tumors of circulating cells. Malignancies of other tissues or organs may produce solid tumors. In general, the methods of the present invention may be used in the treatment of lymphatic cells, circulating immune cells, and solid tumors.

As used to describe the present invention, a “pathogenic virus” is a virus causing disease in a host. The pathogenic virus infects cells of the host animal and the consequence of such infection is a deterioration in the health of the host.

As used herein the term “10 ̂y” is equivalent to 10^{y}. Accordingly, 2.5×10 ̂6 is equivalent to 2.5×10⁶, or 2,500,000.

As used herein, the term “treatment facility” refers to any facility, hospital, clinic, or other institution where the cells are to be shipped. In a preferred embodiment, the cells are shipped to a facility that treats patients.

As used herein, “method for transport” includes any means of transport from the lab or other facility where the NK-92 cells originate to a treatment facility, including without limitation road transportation, air transportation, railway transportation, and, when appropriate, pedestrian transportation (e.g., walking)

Titles or subtitles may be used in the specification for the convenience of a reader, which are not intended to influence the scope of the present invention. Additionally, some terms used in this specification are more specifically defined below.

### NK-92 Cell Line

The NK-92 cell line has been described by Gong et al. (1994). It is found to exhibit the CD56^{bright}, CD2, CD7, CD11a, CD28, CD45, and CD54 surface markers. It furthermore does not display the CD1, CD3, CD4, CD5, CD8, CD10, CD14, CD16, CD19, CD20, CD23, and CD34 markers. Growth of NK-92 cells in culture is dependent upon the presence of recombinant interleukin 2 (rIL-2), with a dose as low as 1 IU/mL being sufficient to maintain proliferation. IL-7 and IL-12 do not support long-term growth, nor do other cytokines tested, including IL-1α, IL-6, tumor necrosis factor a, interferon α, and interferon γ. NK-92 is highly effective in killing certain tumor cells, such as K562 (erythroleukemia) and Daudi (Burkitt lymphoma) cells, for it has high cytotoxicity even at a low effector:target (E:T) ratio of 1:1. Gong, et al., supra. In addition, NK-92 cells have high cytotoxic activity against 8E5 cells, which are infected with HIV and produce HIV virions. NK-92 cells are deposited with the American Type Culture Collection (ATCC), designation CRL-2407.

NK-92 cells demonstrate lytic activity against a broad range of malignant target cells. These include cell lines derived from circulating target cells such as acute and chronic lymphoblastic and myelogenous leukemia, lymphoma, myeloma, melanoma, as well as cells from solid tumors such as prostate cancer, neuroblastoma, and breast cancer cell lines. This effect is observed even at very low effector:target ratios. This lysis is superior to cytotoxicity obtained from normal peripheral blood mononuclear cells stimulated for four days with IL-2. Early-stage clinical studies utilizing irradiated NK-92 cells in patients have indicated good tolerance with an indication of beneficial effects. These studies have been done in patients with a variety of cancer types, with encouraging results in, for example, renal cell cancer and myeloma (Arai et al., Cytotherapy 10:625-632, 2008) and lung cancer (Tonn et al., J. Hematother. & Stem Cell Res. 10:535-544, 2001).

### Compositions and Methods

Some embodiments generally relate to storage mediums, including medium for transport and/or delivery of NK92 cells, having one or more of the parameters as described herein. In one aspect, the invention described herein relates to a sterile, isotonic NK-92 cell storage medium for transport and delivery of NK-92 cells, the storage medium comprising: human serum or human serum albumin; an initial density of non-irradiated, substantially non-aggregated NK-92 cells sufficient to provide a therapeutic amount of NK-92 cells at the time of delivery to a treatment facility; and an IL-2 concentration of about 200 IU/mL, wherein said medium is maintained within +/−5° C. of a temperature selected for transport, and further wherein said temperature selected for transport is between about 20° C. to about 40°C., in the presence of sufficient oxygen to maintain viability of the cells, such that the NK-92 cells remain viable for administration to a patient up to a period of at least 24 hours after placement into the storage medium.

In another aspect, the invention described herein relates to a method for transporting non-irradiated, substantially non-aggregated NK-92 cells to maintain viability of said NK-92 cells for administration to a patient for up to a period of at least 24 hours after placement into a storage medium, the method comprising:

(a) providing a sterile storage container;

(b) adding sterile storage medium to said container, wherein said medium comprises human serum or human serum albumin, and about 200 IU/mL of IL-2;

(c) adding to the container an initial density of non-irradiated, substantially non-aggregated NK-92 cells sufficient to provide a therapeutic amount NK-92 cells at the time of delivery to a treatment facility;

(d) sealing the container;

(e) maintaining the medium within +/−5° C. of a temperature selected for transport, wherein said temperature selected for transport is between about 20° C. to about 40° C.; and

(f) transporting the NK-92 cells for up to a period of at least 24 hours after placement into the storage medium, in the presence of sufficient oxygen to maintain viability of the cells,

such that the NK-92 cells remain viable for administration to a patient for up to a period of at least 24 hours after placement into the storage medium.

In some embodiments, the density of NK-92 cells is maintained between about 1×10 ̂4 cells/mL to about 1×10 ̂7 cells/mL during transport. In some embodiments, the NK-92 cell density is maintained between about 0.5×10 ̂5 cells/mL to about 1×10 ̂7 cells/mL during transport. In some embodiments, the NK-92 cell density is maintained between about 1×10 ̂5 cells/mL to about 1×10 ̂7 cells/mL during transport. In some embodiments, the NK-92 cell density is maintained between about 1×10 ̂6 cells/mL to about 1×10 ̂7 cells/mL during transport. In some embodiments, the NK-92 cell density is maintained between about 2×10 ̂5 cells/mL to about 1×10 ̂7 cells/mL during transport. In some embodiments, the NK-92 cell density is maintained between about 1×10 ̂4 cells/mL to about 9×10 ̂6 cells/mL during transport. In some embodiments, the NK-92 cell density is maintained between about 1×10 ̂4 cells/mL to about 8×10 ̂6 cells/mL during transport. In some embodiments, the NK-92 cell density is maintained between about 1×10 ̂4 cells/mL to about 6×10 ̂6 cells/mL during transport. NK-92 cells may be maintained at any density within any of these ranges, including endpoints.

In some embodiments, the initial density of NK-92 cells is between about 0.5×10 ̂4 cells/ml and about 9×10 ̂6 cells/ml. In some embodiments, the initial density of NK-92 cells is between about 1×10 ̂5 cells/ml and about 9×10 ̂6 cells/ml. In some embodiments, the initial density of NK-92 cells is between about 1×10 ̂6 cells/ml and about 9×10 ̂6 cells/ml. In some embodiments, the initial density of NK-92 cells is between about 1×10 ̂6 cells/ml and about 8×10 ̂6 cells/ml. In some embodiments, the initial density of NK-92 cells is between about 1×10 ̂6 cells/ml and about 7×10 ̂6 cells/ml. In some embodiments, the initial density of NK-92 cells is between about 1×10 ̂6 cells/ml and about 6×10 ̂6 cells/ml. The initial density of NK-92 cells may be any density within any of these ranges, including endpoints.

In some embodiments, the temperature selected for transport is between about 20° C. and about 40° C. In a preferred embodiment, the temperature selected for transport is between about 25° C. and about 38° C. In some embodiments, the temperature selected for transport is between about 20° C. and about 25° C. In some embodiments, the temperature selected for transport is about room temperature. In some embodiments, the temperature selected for transport is about 37° C.

In some embodiments, the medium is maintained within +/−5° C. of a temperature selected for transport. In some embodiments, the medium is maintained within +/−4° C. of a temperature selected for transport. In some embodiments, the medium is maintained within +/−3° C. of a temperature selected for transport. In some embodiments, the medium is maintained within +/−2° C. of a temperature selected for transport. In some embodiments, the medium is maintained within +/−1° C. of a temperature selected for transport.

In some embodiments, a temperature control device maintains the storage medium within +/−5° C. of the temperature selected for transport. Such temperature control devices are known in the art. Exemplary temperature control devices include gel packs that are heated to a desired temperature, portable shipping incubators, bioreactors, or any other suitable device known in the art.

In some embodiments, the NK-92 cells remain viable for administration to a patient up to a period of at least 24 hours after placement into the storage medium. In some embodiments, the NK-92 cells remain viable for administration to a patient up to a period of about 14 hours after placement into the storage medium. In some embodiments, the NK-92 cells remain viable for administration to a patient up to a period of about 20, 16, 12, 10, 9, 8, 7, 6, 5, 4, 3, 2, or 1 hours after placement into the storage medium. In some embodiments, the NK-92 cells remain viable for administration to a patient for a period of greater than 24 hours after placement into the storage medium. In some embodiments, the NK-92 cells remain viable for administration to a patient up to a period of about 36, 48, 60, or 72 hours after placement into the storage medium.

In some embodiments, NK-92 cells may be stored for a period of time after arrival at the treatment facility. For example, the NK-92 cells may be stored and/or grown at the facility before implementation of procedures to inject the NK-92 cells into the patient. In one embodiment, the storage medium is replaced with growth medium (for example, containing a higher concentration of IL-2) after delivery. In one embodiment, additional medium is added to the NK-92 cells for storage; that is, the density of the NK-92 cells is reduced. In one embodiment, the NK-92 cells are stored for 12 hours, 24 hours, 36 hours, or 48 hours after delivery to the facility. In one embodiment, storage conditions of the NK-92 cells depend on cell density, cell growth, cell viability, levels of IL-2, or any combination thereof.

In some embodiments, the medium comprises human serum or equivalent thereof. In some embodiments, the medium comprises human serum albumin. In some embodiments, the medium comprises human plasma. In some embodiments, the medium comprises about 1% to about 15% human serum or human serum equivalent. In some embodiments, the medium comprises about 1% to about 10% human serum or human serum equivalent. In some embodiments, the medium comprises about 1% to about 5% human serum or human serum equivalent. In a preferred embodiment, the medium comprises about 2.5% human serum or human serum equivalent. In some embodiments, the serum is human AB serum. In some embodiments, a serum substitute that is acceptable for use in human therapeutics is used instead of human serum. Such serum substitutes may be known in the art, or developed in the future. Although concentrations of human serum over 15% can be used, it is contemplated that concentrations greater than about 5% will be cost-prohibitive.

The storage container used in the invention described herein can be any cell storage container. In some embodiments, the storage container can be any storage container known in the art. In some embodiments, the storage container is an oxygen-permeable bag. Is some embodiments, the storage container is a tissue culture flask. In some embodiments, the storage container is an oxygen-permeable flask. An example of such a flask is the G-Rex flask, manufactured by Wilson Wolf Manufacturing. In some embodiments, the storage container is a non-oxygen-permeable bag with sufficient oxygen in the headspace to maintain viability of the cells during transport.

In some embodiments, the storage container is labeled with information. In a preferred embodiment, the storage container is labeled with information about the date and time the NK-92 cells were placed in the storage medium. Methods of labeling containers are well known in the art. In some embodiments, the label is a bar code. In some embodiments, the label is a radio frequency identification (RFID) tag. In some embodiments, the label is a two-dimensional bar code. In some embodiments, the label is a quick response (QR) code. In some embodiments, the label is written, typed, or stamped. In some embodiments, multiple labeling methods are employed.

The following abbreviations are used in this application:

| Abbreviation | Definition |
|---|---|
| E:T | Effector to target ratio |
| IL-2 | Interleukin 2 |
| IU | International Units |
| mL | Milliliters |
| SM | Storage medium |

A storage medium (SM) for transport of NK-92 cells is prepared in an appropriate sterile container as indicated in the following table. The medium is filtered using a 0.2 micron PES filter unit. IL-2 (200 IU/mL) is added to the medium to make the SM. In a preferred embodiment, IL-2 is added immediately before use.

| | Volume | | | | |
|---|---|---|---|---|---|
| Reagents | in 1 Liter | 800 ml | 400 ml | 200 ml | 100 ml |
| L-Asparagine, | 0.60 ml | 0.48 ml | 0.24 ml | 0.12 ml | 0.06 ml |
| 60 mM (100X) | | | | | |
| L-Glutamine, | 2.25 ml | 1.80 ml | 0.90 ml | 0.45 ml | 0.23 ml |
| 200 mM | | | | | |
| L-Serine, | 1.80 ml | 1.44 ml | 0.72 ml | 0.36 ml | 0.18 ml |
| 180 mM (100X) | | | | | |
| HI Human Serum | 50.00 ml | 40.00 ml | 20.00 ml | 10.00 ml | 5.00 ml |
| X-VIVO-10 | 945.00 ml | 756.00 ml | 378.00 ml | 189.00 ml | 94.50 ml |
| Medium | | | | | |

Although IL-2 is added above at a concentration of about 200 IU/mL, it should be understood that other concentrations can be utilized, including those described elsewhere herein.

The following examples are included to illustrate the invention and not to limit the invention. All publications or references cited in the present specification are hereby incorporated by reference.

### EXAMPLES

### Example 1

### Doubling Time of NK-92 Cells

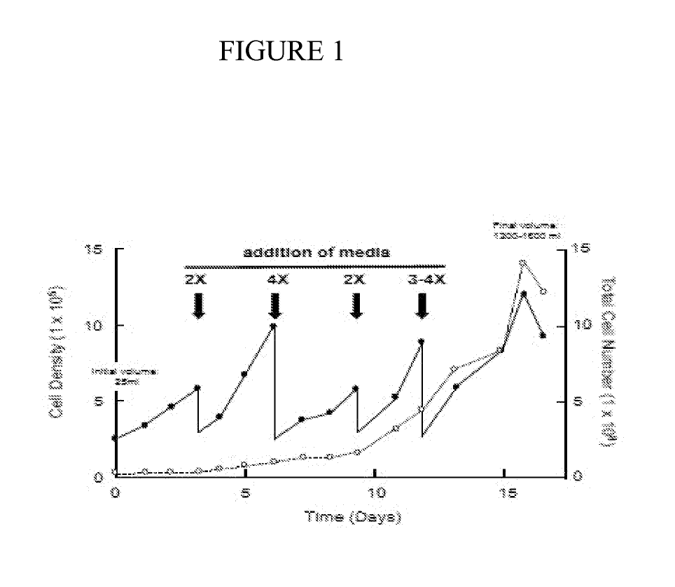

NK-92 cells were cultured in VueLife® culture bags (American Fluoroseal Corp.) in X-VIVO 10 cell culture medium (Lonza, Inc.) supplemented with 2.5% human AB plasma, 500 IU/mL IL-2, asparagine, glutamine, and serine for two weeks. Supplemented cell culture medium was added every 3 days, as indicated in FIG. 1 (2×: two volumes of medium added; 4×: four volumes of medium added; 3-4×: three to four volumes of medium added). Cell density (closed circles) and total cell number (open circles) were determined by cell counting. Doubling time was determined to be between 26 and 32 hours.

### Example 2

### Cytoxicity of NK-92 Cells—Storage/Shipping Cell Density

NK-92 cells were shipped at different cell concentrations in G-Rex 10 flasks containing SM with 450 IU IL-2 on 37° C. pre-heated temperature control packs. The cytotoxic activity of NK-92 cells against K562 cells was determined. K562 (erythroleukemia) cell line was obtained from ATCC and maintained in continuous suspension culture in RPMI 1640 medium supplemented with 10% fetal calf serum (FCS). The cytotoxic activity of NK-92 (effector, E) against K562 (target, T) cells was assessed by means of a ⁵¹Cr release assay using various E:T ratios, as described in Gong, et al. (1994), supra, Klingemann, et al. (Cancer Immunol. Immunother. 33:395-397 (1991)), and U.S. patent application Ser. No. 10/456,237, all three of which are incorporated herein by reference in their entireties. Cytotoxicity was determined either immediately after shipment, or after the cells were diluted to 1×10 ̂6 cells/mL and allowed to rest overnight.

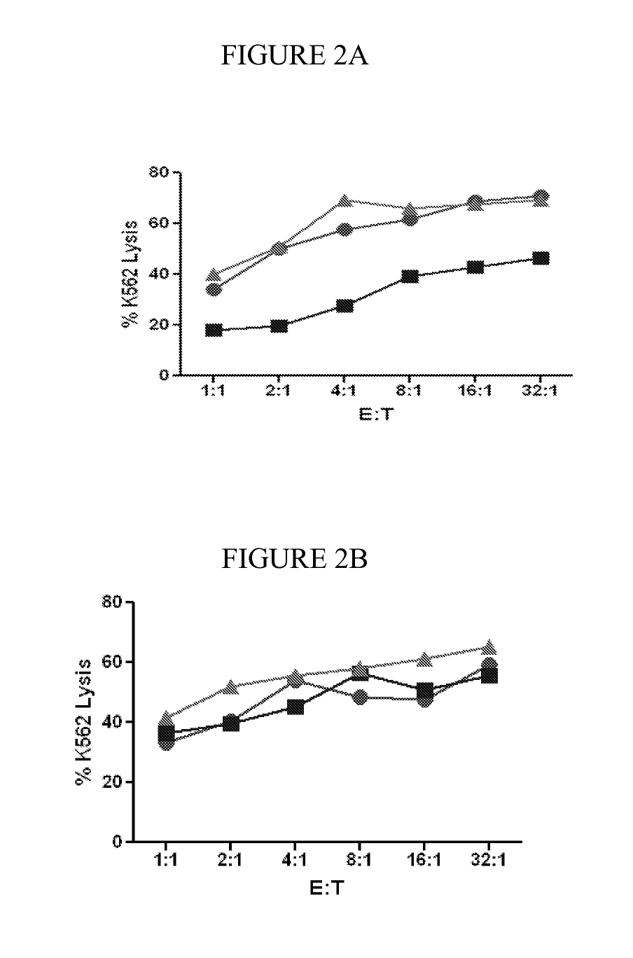

FIG. 2A indicates the cytotoxicity of NK-92 cells shipped overnight at a density of 1×10 ̂6 cells/mL (red circles) or 6×10 ̂6 cells/mL (blue squares), or stored overnight in an incubator at a density of 4×10 ̂5 cells/mL (green triangles). FIG. 2B indicates the cytotoxicity of the cells after dilution and resting. Cytotoxicity of cells shipped at higher density (6×10 ̂6 cell/mL) dropped immediately after shipment, but could be restored when cells were diluted to 1×10 ̂6 cell/mL and rested overnight.

### Example 3

### NK-92 Cell Culturing

NK-92 cells are transported at different cell concentrations in containers containing SM with 200 IU IL-2 pre-heated temperature control packs (e.g., 37° C.). The cytotoxic activity of NK-92 cells against target cells is assessed, for example, by the assay described in Example 2. Cytotoxicity is determined either immediately after shipment, or after the cells are diluted to 1×10 ̂6 cells/mL and allowed to rest overnight.

### Example 4

### Shipping of NK-92 Cells

NK-92 cells were shipped from Texas to Pittsburg. The cells were formulated in GM-1 medium with IL-2 at 1×10 ̂6 cells/ml in one G-Rex 10 (40×10 ̂6 total cells in 40 ml volume). Cells were shipped with temperature-control packs pre-warmed to 37° C. Cells at the time of shipment were 94.9% viable.

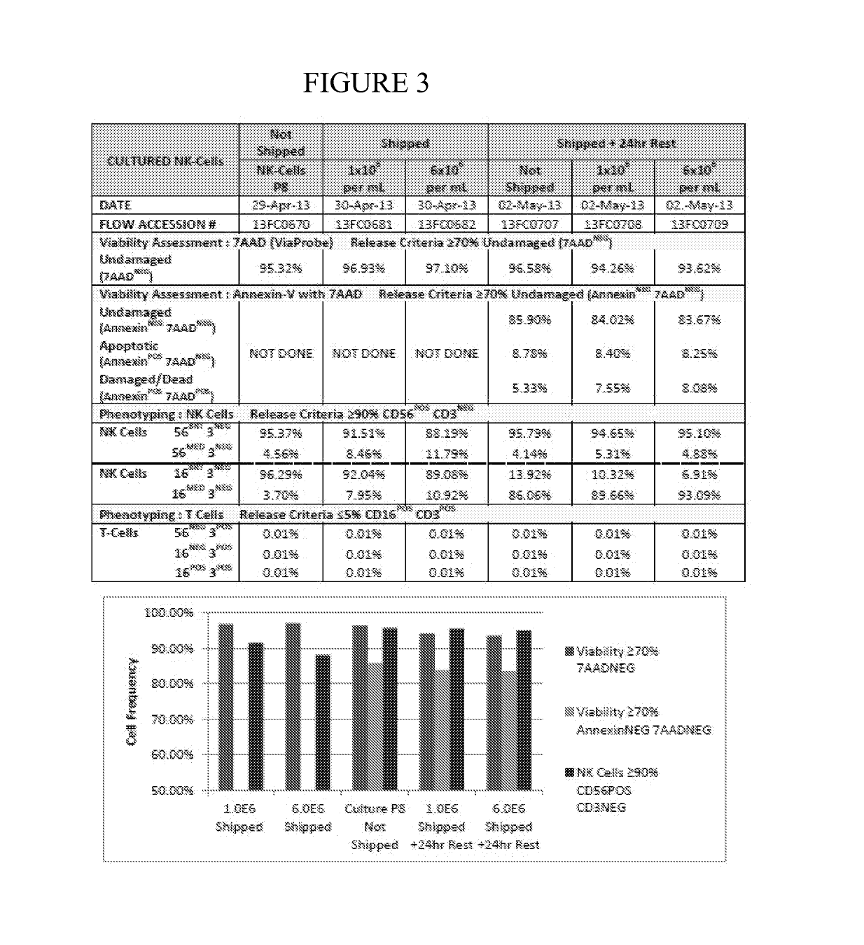

NK-92 cells shipped at 6×10 ̂6 cells/ml were compared to those shipped at 1×10 ̂6 cells/ml by flow cytometry analysis for viability and phenotype. FIG. 3 indicates the results of the flow cytometry assay.

What is claimed is:

1. A sterile, isotonic NK-92 cell storage medium for transport and delivery of NK-92 cells, the storage medium comprising:
(i) human serum or human serum albumin;
(ii) an initial cell density of non-irradiated, substantially non-aggregated NK-92 cells sufficient to provide a therapeutic amount of NK-92 cells at the time of delivery to a treatment facility; and
(iii) an IL-2 concentration of about 200 IU/mL,
wherein said medium is maintained within +/−5° C. of a temperature selected for transport, and further wherein said temperature selected for transport is between about 20° C. to about 40° C., in the presence of sufficient oxygen to maintain viability of the cells, such that the NK-92 cells remain viable for administration to a patient up to a period of at least 24 hours after placement into the storage medium.

2. The storage medium of claim 1, wherein the NK-92 cell density is maintained between about 1×10 ̂4 cells/mL to about 1×10 ̂7 cells/mL during transport.

3. The storage medium of claim 3, wherein the NK-92 cell density is maintained between about 0.5×10 ̂6 cells/mL to about 8×10 ̂6 cells/mL during transport.

4. The storage medium of claim 1, wherein the initial cell density is between about 1×10 ̂6 cells/ml and 6×10 ̂6 cells/ml.

5. The storage medium of claim 1, wherein said medium is maintained within +/−2° C. of the temperature selected for transport.

6. The storage medium of claim 1, wherein said medium comprises about 1% to about 5% human serum or human serum albumin.

7. The storage medium of claim 1, wherein said temperature selected for transport is between about 25° C. to about 38° C.

8. A method for transporting non-irradiated, substantially non-aggregated NK-92 cells to maintain viability of said NK-92 cells for administration to a patient for up to a period of at least 24 hours after placement into a storage medium, the method comprising:
(a) providing a sterile storage container;
(b) adding sterile storage medium to said container, wherein said medium comprises human serum or human serum albumin and about 200 IU/mL of IL-2;
(c) adding to the container an initial density of non-irradiated, substantially non-aggregated NK-92 cells sufficient to provide a therapeutic amount of NK-92 cells at the time of delivery to a treatment facility;
(d) sealing the container;
(e) maintaining the medium within +/−5° C. of a temperature selected for transport, wherein said temperature selected for transport is between about 20° C. to about 40° C.; and
(f) transporting the NK-92 for up to a period of at least 24 hours after placement into the storage medium, cells in the presence of sufficient oxygen to maintain viability of the cells,
such that the NK-92 cells remain viable for administration to a patient for up to a period of at least 24 hours after placement into the storage medium.

9. The method of claim 8, wherein the NK-92 cell density is maintained between about 1×10 ̂4 cells/mL to about 1×10 ̂7 cells/mL during transport.

10. The method of claim 9, wherein the NK-92 cell density is maintained between about 0.5×10 ̂6 cells/mL to about 8×10 ̂6 cells/mL during transport.

11. The method of claim 8, wherein the initial density is between about 1×10 ̂6 cells/ml and 6×10 ̂6 cells/ml.

12. The method of claim 8, wherein said medium is maintained within +/−2° C. of the temperature selected for transport.

13. The method of claim 8, wherein a temperature control device maintains the storage medium within +/−5° C. of the temperature selected for transport.

14. The method of claim 8, wherein the storage container is labeled with the date and time the NK-92 cells were added to the storage medium.

15. The method of claim 8, wherein said medium comprises about 1% to about 5% human serum or human serum albumin.

16. The method of claim 8, wherein said temperature selected for transport is between about 25° C. to about 38° C.
